(19) United States
(12) Patent Application Publication
(10) Pub. No.: US 2024/0307206 A1
(43) Pub. Date: Sep. 19, 2024

(54) Foot Drop Assistive Device

(71) Applicant: John Douglas Bruno, Mount Laurel, NJ (US)

(72) Inventor: John Douglas Bruno, Mount Laurel, NJ (US)

(21) Appl. No.: 18/607,204

(22) Filed: Mar. 15, 2024

Related U.S. Application Data
(60) Provisional application No. 63/490,704, filed on Mar. 16, 2023.

(51) Int. Cl. A61F 5/01

(52) U.S. Cl. CPC A61F 5/0113

(57) ABSTRACT

An assistive device for preventing foot drop. The assistive device is worn around the leg and torso. An elastic cord connects the foot to a torso strap. The clastic cord wraps behind the knee to direct the tensional force in the clastic cord in the proper direction to lift the foot. A leg strap secures the clastic cord behind the knee. The torso strap may include a shoulder pad for comfort.

### FIELD OF THE INVENTION

The present invention relates generally to mobility assistive devices. The present invention relates more specifically wearable devices to prevent foot drop.

### BACKGROUND OF THE INVENTION

A number of medical conditions can lead to difficulty or inability to lift the foot, a condition known as “foot drop.” Foot drop causes the foot the drag on the floor when walking. A person suffering from foot drop frequently has difficulty walking and may frequently stumble due to an inability to lift the foot above obstacles. Existing solutions tend to lock the knee, preventing natural flexing of the knee while walking.

It is an objective of the present invention to provide an assistive device to aid in lifting the foot. It is a further objective of the present invention to provide an assistive device that assists with knee flexing and hip flexing. It is a further objective of the present invention to provide an assistive device that can be worn under clothing or above clothing, according to preference.

### SUMMARY OF THE INVENTION

The present invention comprises an assistive device that is worn around the leg and torso. The device aids in lifting the front of the foot and the leg while walking. The assistive device is worn around the leg and torso. An elastic cord connects the foot to a torso strap. The elastic cord wraps behind the knee to direct the tensional force in the elastic cord in the proper direction to lift the foot. A leg strap secures the elastic cord behind the knee. The torso strap may include a shoulder pad for comfort. The assistive device is connected to the shoe to provide lifting force to the foot.

### BRIEF DESCRIPTION OF THE DRAWINGS

FIG. 1 is a perspective view of Embodiment 1 of the present invention.

FIG. 2 is a front elevation view of Embodiment 1 of the present invention.

FIG. 3 is a rear elevation view of Embodiment 1 of the present invention.

FIG. 4 is a perspective view of Embodiment 2 of the present invention.

### DETAILED DESCRIPTION OF THE INVENTION

All illustrations of the drawings are for the purpose of describing selected versions of the present invention and are not intended to limit the scope of the present invention.

The present invention provides an assistive device that directs forces around three critical points: the front of the foot, the back of the knee, and the shoulder. The assistive device lifts the front of the foot towards the knee. The assistive device redirects the tension in the device around the back of the knee and upward towards the shoulder. The shoulder provides a lifting force to the device. The tension between the shoulder and the knee helps lift the thigh while walking. As the wearer walks, the lifting force on the foot lifts the foot with minimal or no muscular force from the wearer.

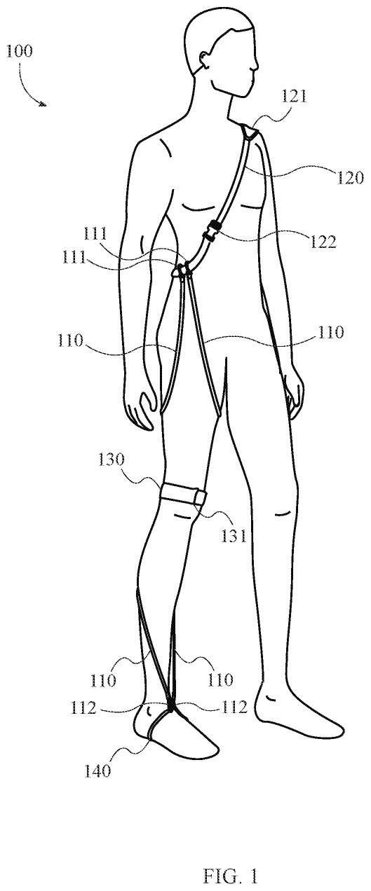

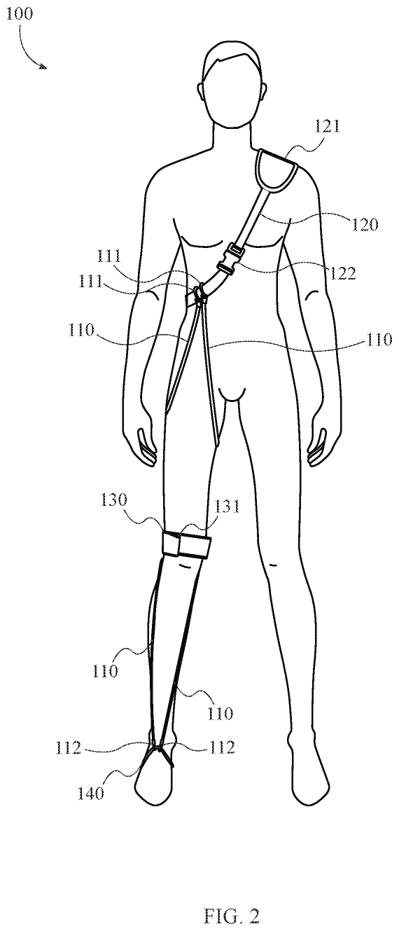

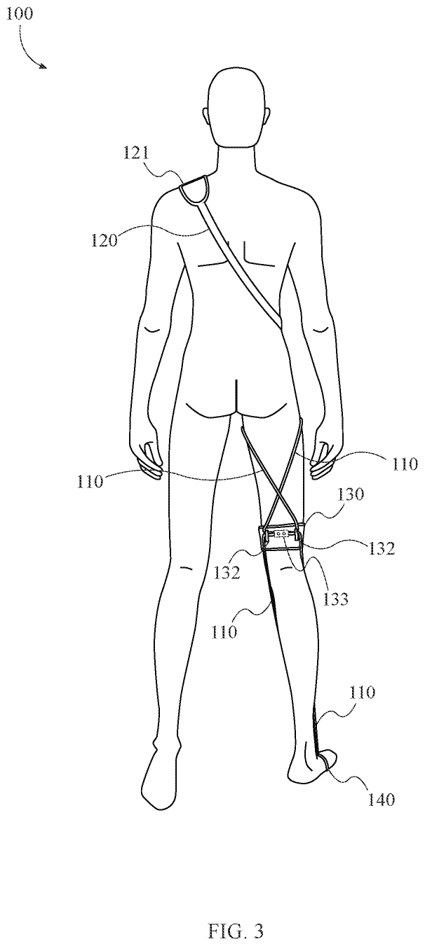

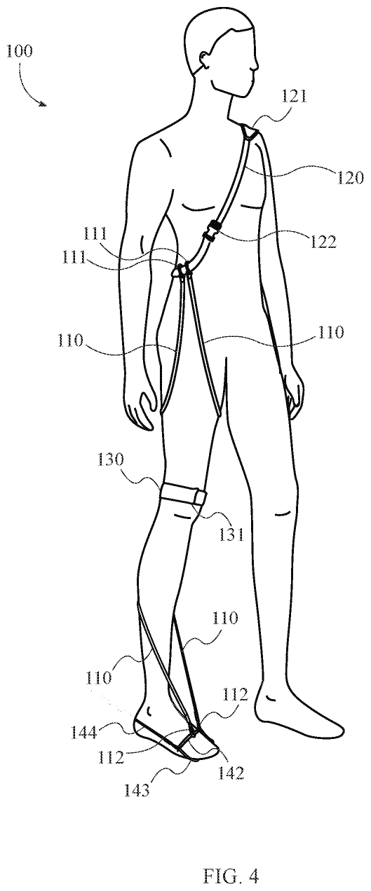

Referring to FIGS. 1-4, the present invention comprises an assistive device 100. The assistive device 100 comprises elastic cords 110, a torso strap 120, and a leg strap 130. The elastic cords wrap around the leg of the wearer. Embodiment 1 comprises two elastic cords 110, wrapping around the leg in opposite directions. Alternate embodiments may include a single elastic cord or more than two elastic cords. The ends of the elastic cords 110 proximate to the torso include loops 111. The torso strap 120 traverses through the loops 111 creating a secure connection between the torso strap 120 and the elastic cords 110. The elastic cords 110 provide a tensional force between the foot and the back of the knee and a tensional force between the back of the knee and the torso strap 120. The tensional force between the foot and the back of the knee lifts the foot while the wearer is walking. The torso strap 120 transfers the tensional force from the elastic cords 110 to the shoulder of the wearer.

The ends of the elastic cords 110 distal from the torso strap 120 include a foot fastener 140. In Embodiment 1, the distal ends of the elastic cords 110 comprise hooks 112. The foot fastener 140 comprises a foot loop that traverses the hooks 112 to create a secure connection between the elastic cords 110 and the foot loop. The foot loop encircles the foot of the wearer and provides a lifting force to the foot. In Embodiment 2, the foot fastener comprises an upper toe strap 142, a lower toe strap 143, and a heel strap 144. Each of the toe straps has a roughly “C” shape. The upper toe strap 142 is connected to the lower toe strap 143 at each end. The intersection of the toe straps forms a rough “V” shape. The heel strap 144 connects to the toe straps adjacent to the intersection of the toe straps. In an example, each toe strap is constructed from a zip tie with a loop adjacent to the lock. In this example, the heel strap 144 is threaded through the loop to connect the heel strap 144 to the toe straps. In Embodiment 2, the distal ends of the elastic cords 110 comprise hooks 112. The hooks 112 connect to the upper toe strap 142. In Embodiment 3, zip ties connect the hooks 112 at the distal ends of the elastic cords 110 to the shoelaces of the wearer's shoe. In Embodiment 4, the elastic cords 110 connect to a strap that is inserted through the eyelets in the shoes of the wearer.

The torso strap 120 encircles the torso of the wearer. The torso strap includes a shoulder pad 121 and a buckle 122. The shoulder pad 121 provides a cushion to enhance comfort for the wearer. The buckle 122 provides an easier mechanism for donning and doffing the assistive device 100. The buckle 122 includes a length adjustment mechanism that allows the wearer to adjust the length of the torso strap 120. Adjusting the length may enhance wearer comfort and allows the wearer to adjust the tension in the elastic cords 110, thereby increasing or decreasing the lifting force on the foot and/or thigh. For example, the wearer may use the torso strap 120 to make on-the-go adjustments to the tension in the elastic cords.

The leg strap 130 encircles the leg of the wearer. The leg strap 130 may include a fastener 131 that allows the wearer to adjust the length of the leg strap 130. In Embodiment 1, the fastener 131 is a hook and loop fastener. The leg strap 130 includes loops 132 and clamp 133. In Embodiment 1, the leg strap 130 includes two loops 132. The clamp 133 is disposed between the two loops 132. In alternate embodiments, a single loop or more than two loops may be used. The elastic cords 110 traverse through the loops 132 and traverses through the clamp 133. The clamp 133 secures the elastic cords 110, preventing the elastic cords 110 from moving relative to one another. In Embodiment 1, the clamp 133 is closed with screws. The screws may be loosed to adjust the elastic cords 110, and the screws may be tightened to secure the elastic cords 110. For example, the wearer may use the clamp 133 to adjust the elastic cords 110 before donning the assistive device. The leg strap 130 secures the elastic cords 110 to prevent the elastic cords 110 from shifting around the leg during use. The leg strap 130 provides an anchor point to redirect the tensional forces of the elastic cords 110. The position and tightness of the leg strap 130 can be adjusted to adjust the tension in the elastic cords 110. The leg strap 130 helps maintain the proper distribution of tensional forces in the elastic cords 110 to aid the wearer when walking.

To use the assistive device 100, the foot fastener 140 is attached to the shoe or foot of the wearer. Next, the leg strap 130 is strapped on the wearer's thigh, just above the knee. The elastic cords 110 are adjusted to achieve the desired tension. The clamp 133 is tightened to lock the elastic cords 110 in place relative to each other. This adjustment of the elastic cords 110 and clamp 133 is used to make coarse adjustments to the elastic cords 11. The torso strap 120 is then placed around the torso of the wearer. The length and position of the torso strap 120 is adjusted to make fine adjustments to the elastic cords 110.

Although the invention has been explained in relation to its preferred embodiment, it is to be understood that many other possible modifications and variations can be made without departing from the spirit and scope of the invention as hereinafter claimed.

What is claimed is:

1. An assistive device comprising:
a plurality of elastic cords;
a torso strap;
a leg strap;
a foot fastener;
the elastic cord connected to the torso strap;
the elastic cord connected to the foot fastener; and
the leg strap connected to the elastic cord.

2. The assistive device of claim 1, wherein:
the leg strap further comprises a clamp; and
the clamp securing the plurality of elastic cords.

3. The assistive device of claim 2, wherein:
the leg strap further comprises two or more loops;
the elastic cord traversing the two or more loops; and
and the clamp disposed between the two or more loops.

4. The assistive device of claim 1, wherein the leg strap further comprises a hook and loop fastener.

5. The assistive device of claim 1, wherein:
the elastic cord further comprises a loop;
the loop being connected to a proximate end of the elastic cord; and
the torso strap traversing the loop.

6. The assistive device of claim 1, wherein the leg strap further comprises a hook and loop fastener.

7. The assistive device of claim 1, wherein the foot fastener further comprises:
an upper toe strap;
a lower toe strap;
a heel strap;
the heel strap connected to the upper toe strap; and
the heel strap connected to the lower toe strap.

8. The assistive device of claim 7, wherein the upper toe strap connects to a distal end of the plurality of elastic cords.

9. The assistive device of claim 1, wherein:
the foot fastener further comprises a loop; and
the loop being connected to a distal end of the plurality of elastic cords.

10. The assistive device of claim 1, wherein:
the foot fastener further comprises one or more zip ties; and
the one or more zip ties being connected to a distal end of the plurality of elastic cords.

11. The assistive device of claim 1, further comprising:
a shoulder pad; and
the shoulder pad being connected to the torso strap.

12. The assistive device of claim 1, wherein the torso strap further comprises a buckle.

13. The assistive device of claim 12, wherein the buckle further comprises a length adjustment mechanism.

14. An assistive device, comprising:
a torso strap;
a plurality of elastic cords;
a leg strap;
a shoulder pad;
a buckle;
a clamp;
a foot fastener;
each of the plurality of elastic cords further comprising a loop fastener at a proximate end of the plurality of elastic cords;
the torso strap traversing the loop fastener of each of the plurality of elastic cords;
the foot fastener connected to a distal end of the plurality of elastic cords;
the leg strap further comprising a plurality of loops;
the plurality of elastic cords traversing the plurality of loops of the leg strap; and
the clamp securing the plurality of elastic cords.

15. The assistive device of claim 14, wherein the foot fastener further comprises:
an upper toe strap;
a lower toe strap;
a heel strap;
the upper toe strap connected to the lower toe strap
the heel strap connected to the upper toe strap; and
the heel strap connected to the lower toe strap.

16. The assistive device of claim 15, wherein the upper toe strap connects to the distal end of the plurality of elastic cords.

17. The assistive device of claim 14, wherein the buckle further comprises a length adjustment mechanism.

18. The assistive device of claim 14, wherein the leg strap further comprises a hook and loop fastener.

19. The assistive device of claim 14, wherein:
the foot fastener further comprises a loop; and
the loop being connected to a distal end of the elastic cord.

20. The assistive device of claim 14, wherein:
the foot fastener further comprises one or more zip ties; and
the one or more zip ties being connected to a distal end of the elastic cord.
